(19) United States
(12) Patent Application Publication
(10) Pub. No.: US 2015/0290029 A1
(43) Pub. Date: Oct. 15, 2015

(54) Delivery Systems for Agents to treat Ocular Discomfort

(71) Applicant: Edward Tak Wei, Berkeley, CA (US)

(72) Inventor: Edward Tak Wei, Berkeley, CA (US)

(21) Appl. No.: 13/999,979

(22) Filed: Apr. 11, 2014

(51) Int. Cl. A61F 9/00; A61K 9/00; A61K 31/66

(52) U.S. Cl. CPC A61F 9/0008; A61K 31/66; A61K 9/0048

(57) ABSTRACT

Eye drops are the traditional method for delivering drugs to the ocular surface but this method is considered inefficient and does not work well for soothing agents because sensory discomfort is evoked from the corneal surface by the pulsatile delivery of the agent. By contrast, eye wipes moistened with an evenly dispersed liquid composition of a soothing or cooling agent selectively delivers the active ingredient to receptors in the skin above the eyes, on the edges of the eyelids, and on the conjunctiva and do not evoke eye irritation. The delivered agent is now able to evoke sensations of refreshment and soothing on the ocular surface and to relieve eye discomfort.

### RELATED APPLICATIONS

This application is a continuation-in-part of a National Stage application filed Feb. 9, 2012 as application Ser. No. 13/261,061, which has an International Filing date of Jun. 4, 2010 (International App. No. PCT/GB2010/001094) and has priority from U.S. provisional application 61/217,834 (filed Jun. 5, 2009) and 61/270,214 (filed Jul. 6, 2009), the contents of both of which are incorporated herein by reference in their entirety.

### TECHNICAL FIELD

The present invention pertains generally to the field of ocular treatment, and more specifically to the use of a composition comprising a soothing agent to reduce sensory discomfort. The composition is topically administered to at least a portion of the external surface of the eyelid (preferably the closed eyelid) of the eye to be treated. Preferably, the composition including the soothing agent is carried on or in a swab, wipe, pad, or towelette, for example, an eye swab.

### BACKGROUND

The eye surfaces are exposed to the external environment. These anatomical structures—eyelids, front (anterior) part of the eyeball, conjunctiva, lachrymal system, precorneal film and cornea—are subject to injury by physical, chemical and biological agents. The results of injury to the ocular surface are symptoms of discomfort, defined as blurring of vision, itching, irritation, burning sensations, and pain. The signs of injury in the vascularized portions of the eye are redness, swelling, and increased blood flow. Ophthalmic products such as solutions, ointments, and inserts are used to manage the causes and the symptoms and signs of eye injury.

Ophthalmic solutions, administered onto the eye surface in the form of eyedrops, are the most common form of drug delivery to treat anterior eye disorders. This method is preferred to ointments and inserts because of ease and costs of preparation, patient familiarity with procedures of drug dosing, and the lower frequency of side effects. It is recognized, however, that eyedrops are a relatively inefficient method of delivery because individual eyedrops range from 20 to 50 μl in volume, whereas the precorneal space in normal subjects are about ˜7 μl. The excess volume rolls down the cheek or may be absorbed into the nasolacrimal duct. Eyedrops are also difficult to administer because the patient must be taught to recline their head at a 45 to 55° angle and to manually coordinate delivery while keeping their eyes open. Nevertheless, conventional eyedrops represent approximately 90% of marketed formulations for eye disorders. This can be seen at the pharmacy where eyedrop products for dry eyes (e.g. Systane™) and eye irritation (e.g. Visine™) are on display.

It is known by experience that an ice pack applied to the orbit will relieve the pain and discomfort of eye injury. Systematic studies by Fujishima et al. [Amer. J. Ophthalmol. 119: 301-306, 1195; Cornea 16: 630-634, 1997] showed that cooling will relieve the pain and inflammation in the orbit after cataract surgery. In healthy volunteers, discomfort in the eye induced by pressure on the cornea with a nylon microfilament was much better relieved by artificial tears kept at 4° C. than by tears at 25° C. or 36° C. Thus, cooling is recognized as a potential method for relieving sensory discomfort in the eye. In the patent literature, chemical cooling agents such menthol (in combination with antihistamines) [U.S. Pat. No. 6,147,081], trialkylphosphine oxides [US 20050059639] or p-menthane carboxamides [US 20050137166] have been proposed for use in eyedrops to relieve sensory discomfort. Rohto Pharmaceuticals sells in the United States eyedrops labelled as “Cool”, “Ice”, and “Arctic” to relieve redness and to soothe eye strain. In two of these formulations, the cooling ingredient is most likely menthol, which is listed as an “inactive ingredient.” Menthol is an irritant and, in my experience, the “Ice” and “Arctic” formulations cause an initial stinging sensation in the eye, the intensity of cooling is limited, and the duration of cooling is less than 15 minutes.

An irritated eye surface is a common symptom of many disorders, especially in the condition known as “dry eyes”, which is exacerbated, for example, by a dry climate, an increased use of contact lenses, excessive staring at computer screens, and ageing. The estimated prevalence of dry eyes in the United States is about 10 to 30% of the population over age 40, with about 4.9 million severe cases requiring specific medical treatment. Thus, there is need for innovative technology to improve symptomatic or curative treatment.

In a patent application on new cooling compounds, Wei, US 20080227857, published Sep. 18, 2008, N-Alkylcarbonyl-Amino Acid Ester and N-Alkylcarbonyl-Amino Lactone Compounds and Their Use. described delivery of cooling compounds to the skin using a towelette. The wiping of the towelette across skin results in delivery of dermatologically active ingredient(s), meaning that the skin is substantially medicated.

Eye drops are also a relatively inefficient method of delivery. The volume of individual eye drops in commercial samples varies and ranged from 25 to 56 μL [German, E. J., Hurst, M. a, & Wood, D. (1999). Reliability of drop size from multi-dose eye drop bottles: is it cause for concern? Eye (London, England), 13 (Pt 1)(June), 93-100.] in one study and 34 to 63 μL in another [Sklubalová, Z., & Zatloukal, Z. (2006). Study of eye drops dispensing and dose variability by using plastic dropper tips. Drug development and industrial pharmacy, 32(2), 197-205]. The volume delivered can be influenced by parameters such as the physicochemical characteristics of the formulation, the aperture of the bottle, and the angle of delivery. The precorneal space in normal subjects is about ˜7 μL per eye. The conjunctival spaces, also called “cul-de-sac” for the extended lower eyelid, is ˜30 μL. The delivered drop impacts the eyeball with a residence time of less than 1 min and the excess volume rolls down the cheek or is shunted to and absorbed into the nasolacrimal duct via the lacrimal puncta.

The surfaces of the eyelids, conjunctiva, and cornea contain sensory nerve endings that respond to stimuli that range from touch and pressure, to cooling, and to warmth, and to noxious stimuli. The sensory receptors are located on the nerve endings of branches of the trigeminal nerve: a) the supraorbital nerve innervates the upper eyelids and the conjunctiva, b) the infraorbital nerve innervates the lower eyelids, and c) the long ciliary nerves innervate the cornea. These sensory afferents then project rostrally to brainstem dorsolateral nucleus where afferent nerve impulses are interpreted as sensations from the ocular surfaces.

The signals that evoke afferent nerve discharge from the cornea have been characterized by studies of single unit recordings from the ciliary nerve [Belmonte, C. et al. (2004) Nerves and sensations from the eye surface, Ocul. Surf., 2, 248-253.] About 15% of corneal fibers, all fast conducting Aδ-type, respond exclusively to mechanical force. About 70% of corneal fibers, mostly slow-conducting C-fibers, respond only to nociceptive stimuli (e.g. heat above 39° C., cold below 29° C., and chemical irritants) and are called polymodal nociceptors. A third category of corneal nerve fibers, about 10-15%, are A_{δ} and C fibers that discharge spontaneously at rest and increase their firing rate when the normal temperature of the corneal surface (˜33° C.) decreases by ˜0.1° C. These are the cold-sensitive receptors that respond to “innocuous” cooling, that is in the temperature ranges above 29° C. and below 39° C.

The corneal surface is estimated be ⅙ of the total area of the anterior eyeball. It is densely packed with polymodal nociceptors and is exceptionally sensitive to painful stimuli. Eye drops that contain chemical irritants easily evoke discomfort (burning sensations, itch, and pain) from the corneal surface: two examples being Mydriacyl® and Restasis® containing tropicamide and cyclosporine as the active ingredients, respectively. Menthol and ethanol [and other alcohols] are also recognized eye irritants.

### SUMMARY OF THE INVENTION

The present invention provides a method for topically self-delivering a predetermined dose of a selected drug to an eye via the skin covering the eyeball, the edges of the eyelids and/or the conjunctiva. Using this method of drug delivery that avoids and minimizes contact with the corneal surface, sting and discomfort is reduced. An eye swab or wipe embodiment carrying a preferred drug agent provides a substantial therapeutic improvement over eye drops. Proof of concept with a soothing and cooling agent delivered in this manner is illustrated in the examples.

### BRIEF DESCRIPTION OF THE DRAWINGS

FIG. 1 illustrates a preferred delivery apparatus for ocular drugs in accordance with the invention;

FIGS. 2A-C illustrate the impact of drug delivery onto an eye, while FIG. 2D is a graph of drop position and eye contact time;

FIG. 3 graphically illustrates the response of the ocular surface to a preferred agent (CPS-148) applied with eye wipes in accordance with the invention;

FIG. 4 is a graph of fluorescence (Relative Fluourescence Units; % Maximum) of test compounds, as a function of the logarithm of the concentration of the test compound (μM); and

FIG. 5 is a graph of the activity of 2-5 on TRPA1 and TRPV1 transfected cells. Capsaicin and mustard oil are the positive control substances for TRPV1 and TRPA1, respectively.

### DETAILED DESCRIPTION OF THE INVENTION

Broadly, the invention provides a method for topically delivering a predetermined dose of a selected drug to an eye via the skin covering the eyeball, the edges of the eyelids and/or the conjunctiva. An eye swab or wipe embodiment carrying a preferred drug agent provides a substantial therapeutic improvement over eye drops and can be self-delivered.

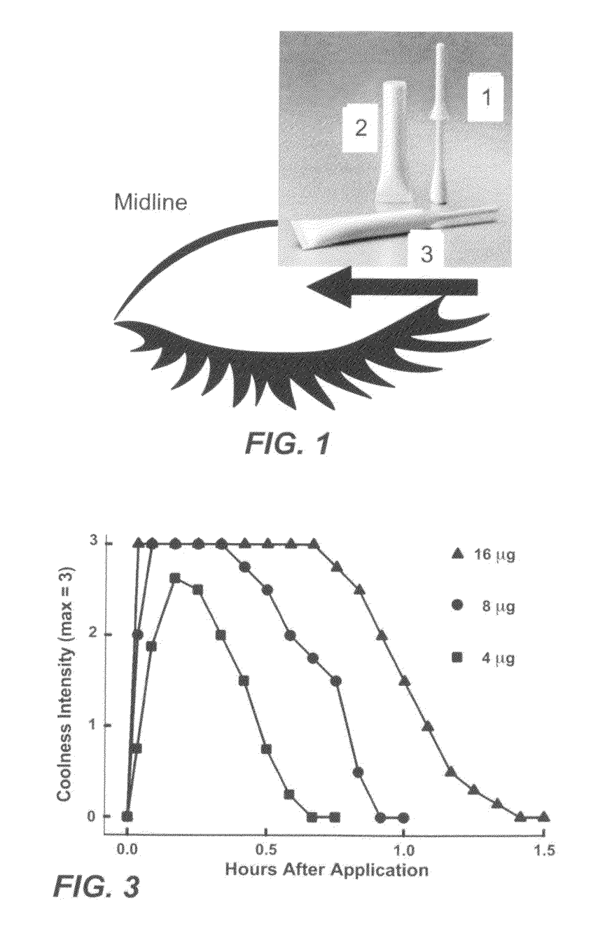

Description of Preferred Delivery Apparatus
1. An applicator preferably is a “swab” in form of a rod (preferably 3 inches) made of plastic (e.g. polystyrene) to which is attached an absorbent material (preferably cotton or rayon) of a predetermined mass (e.g. 40 to 100 mg of cotton). This apparatus is illustrated in FIG. 1. “Delivery apparatus for ocular drugs”. The absorbent material carries the active ingredient which is in a liquid solution. One end of the rod serves as a handle to manipulate the absorbent material to a correct position for application while the other end carries the absorbent material. The dimensions and mass of the absorbent material on the tip of the applicator is critical to the practice of this invention. The absorbent material is saturated because it is bathed in the liquid solution: so the amount of fluid off-loaded is determined by the mass of the absorbent material. Too little, and not enough solution is delivered; too much, and the solution coalesce on the eyelid surface to form drops and runs off the delivery target. By experiment, it was found that a cotton or rayon mass of 40 to 100 mg per tip was optimal for a delivered volume of 8 to 20 μL per eye.
2. A “reservoir” contains the liquid formulation of the active ingredient (e.g. 0.5 to 1.0 mL) sufficient to saturate the absorbent material. The wrapping material of the “reservoir” is not permeable to liquids and may, for example, be cellophane or polystyrene, or other forms of plastic. The reservoir can also be a bottle of solution from which multiple doses may be acquired with individual disposable applicators.
3. The applicator and reservoir can be viewed as a “delivery unit”, which is sealed and may be prepared under clean conditions, or further made sterile (e.g. by gamma irradiation). The delivery unit preferably further includes instructions for use such that the unit and instructions together constitute a kit for use in diagnosing or treating eye disorders.
4. The “delivery method” is to hold the handle end of the swab and to wipe the tip of swab carrying the absorbent material across the lower aspect of the upper eyelid, touching the eyelashes, and moving the swab in a lateral to medial direction, with the eyes closed. Two or more wiping motions are preferred, with a sensation of wetness on the eyeball confirming that the liquid formulation has been delivered. Alternatively, the swab may be dabbed on the lateral canthus with the eyes closed. After application, the subject is instructed to blink. By this method, the preferred delivered volume is 8 to 20 μL per eye.

The advantages of the delivery unit and the delivery method over eye drops are several-fold. The risks of contamination of the applied solution are minimized as there is no direct hand contact with the swab tip. The delivery unit is disposable. The intricacies of hand-eye coordination, angulation of the head, and avoiding accidental contact of the eye drops bottle tip to the eye surface, no longer apply with this method of delivery. The method minimizes the bolus effects of eye drops on the corneal surface and, hence, there is less irritation and discomfort. The distribution of the active ingredient over the ocular surface is more uniform by this swab method of delivery.

### Delivery Unit

Prototypes of the delivery unit are available from manufacturers, for example, SwabDose™ from Unicep Corp. [1702 Industrial Drive, Sandpoint, Id.], and Pro-Swabs from American Empire Manufacturing [3828 Hawthorne Court, Waukegan, Illinois]. These units can be produced en masse, for example in lots of 500,000 units. The specifications of the delivery unit are made to order. Traditionally, these units are used for the delivery of dermatological products, e.g. anti-fungal agents for the toenail. A similar, sophisticated device is made by the S & B. Co., Ltd. Masan-Si, Korea. The apparatus, a “Magic Bar” maintains a solution above a cotton tip by capillary action. Twisting the hollow tube containing the solution allows the liquid to descend on the cotton tip and be ready for delivery.

As shown in the Examples, the substrate on the tip of the applicator must have a certain liquid absorptive capacity to deliver the correct volume, upon wiping of the substrate onto the ocular skin and eyelashes. This was determined by experiment and, surprisingly, approximated the same volume as a small eye drop: namely, ˜30 μL. But the dispersion of the liquid on the target site was quite different from an eye drop.

### Delivery Method

By creating a method of delivery of an agent to the ocular surface by wiping, the delivery procedure is made more convenient than eye drops. Also, by wiping a liquid composition, contact with the cornea is minimized, and the active ingredient is delivered to the ocular surface with less irritation and discomfort.

Drug Delivery Considerations:

Ophthalmic solutions, administered onto the eye surface in the form of eye drops, represent at least 90% of formulations marketed for treatment of anterior eye disorders. The eye drops method is preferred to ointments and inserts because of ease and costs of preparation, patient familiarity with procedures of drug dosing, and the lower frequency of side effects. It is recognized, however, that eye drops are a relatively inefficient method of delivery. Thus, the pulsatile delivery of an eye drop bolus impacts the curved surface of the eye but there is little time for the active ingredient to reach target receptors. The excess volume splashes on the eye surface, rolls down the cheek, or may be absorbed into the nasolacrimal duct. The contact time of the eyedrop with the ocular surface is less than 1 min and washout is further accelerated by the blink reflex and tear turnover.

Fluid Mechanics of the Ocular Surface:

There are several key surfaces on the orbit. The skin, with a contact angle with water of 100 degrees, is mildly hydrophobic, and, like polyethylene, is not easily “wettable” by water. The eyelashes, like skin, are also hydrophobic, and the cylindrical hair shaft increases the surface area for deposition of liquid. Wettability of the eye is achieved using the eye lashes, the mucocutaneous junction, and the eye surface which has a precorneal tear film that is ˜98% water.

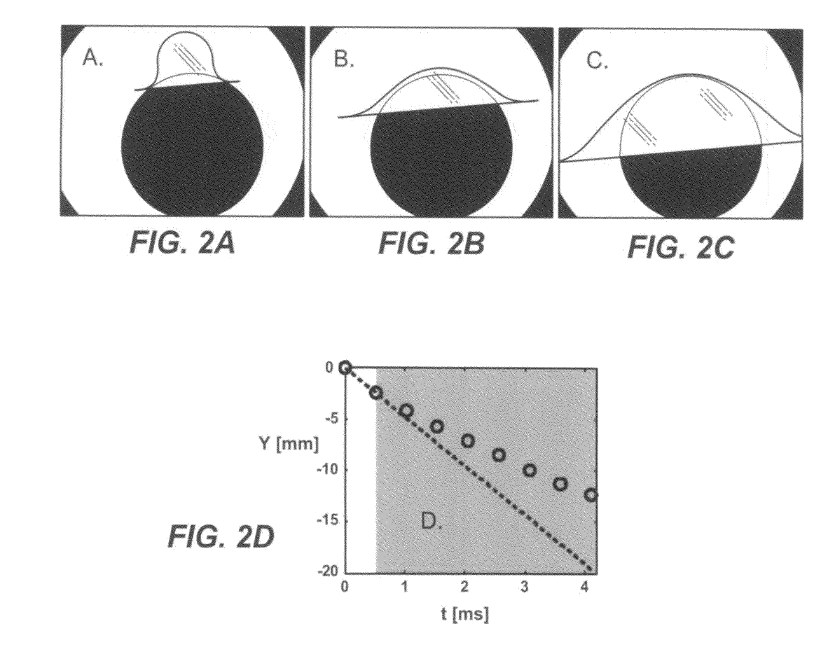

When an aqueous solution is delivered onto the ocular surface, several scenarios can be described.
When an eye drop is applied to the spherical eyeball, the momentum of the drop will cause a lateral splash. See FIG. 2. “Drop impacted at 5 m/s upon an eye mimic of diameter 2 cm. Accompanying graph shows the rapid time course of drop position, wherein the orange-shaded zone indicates the short contact time with the eye” [Figure is obtained through the courtesy of Prof. David Hu, Georgia Institute of Technology and Emory School of Medicine, Atlanta, Ga.]. Using Bernoulli's equation, the height L of the eyedropper above the eye yields the speed of the drop and its accompanying splash, u=(2gL)^{0.5}. Thus, even for modest dropper heights of 5 cm, drops fall at 100 cm/s causing substantial lateral splashes, as observed in FIG. 2, wherein most of the liquid is splashed across the target. The splash event occurs in milliseconds, giving little contact and residence time for the active ingredient on the eye surface.
When the liquid is delivered via an absorbent material (e.g. cotton) on the tip of an applicator and wiped over the eyelid and eyelashes, a much more controlled deposition process is achieved. The use of an applicator enables a thin 0.02 cm layer of fluid to be deposited on the eyelid and eyelashes. This layer is uniform in thickness across the eyelid, which ultimately enables it to be spread more uniformly on the eye than the eye drops. Fluid in the layer is drawn into the eye by a combination of gravitational forces and the wicking action of individual strands of eyelash. The subsequent blink pushes the liquid in a downward concentric direction and the precorneal film, now containing the active ingredients, is swept across the eye surface like a gentle wave over a calm lake. This method takes advantage of the larger surfaces of the eyelid and eyelashes and the natural lubrication processes of the blink to deposit the ingredients uniformly and in appropriate volumes onto the ocular surfaces.

Logic of Delivery by Swabbing or Wiping:

The eye has its own natural wiping mechanisms. Tears, secreted from the lacrimal gland into the upper lateral corner of the eye, are composed ˜98% of water, but also contain biologically active ingredients such as mucins, lipids, proteins such as immunoglobulins and lysozyme, some peptide hormones, and salts. Blinking, the alternate contraction and relaxation of the orbicularis oculi and the levator palpebral superior muscles, close and open the eyelids, respectively. The margins of the eyelids distribute the tears evenly over the ocular surface, and gently push the tear film towards the puncta at the medial canthus for drainage. At the inner lid border, the marginal conjunctiva forms a thickened epithelial cushion that is in closest contact with the bulbar surface, and facilitates distribution of the tears as a thin precorneal film. This thickened cushion has been termed a “lid wiper” [Knop, E., Korb, D. R., Blackie, C. a, & Knop, N. (2010). The lid margin is an underestimated structure for preservation of ocular surface health and development of dry eye disease. Developments in ophthalmology, 45, 108-22.] and is analogous to the edges of a wiper blade on the windshield of the car. By analogy, the added ingredients in tears may be viewed as supplements in the windshield wiper fluids.

The delivery method described here utilizes the eyelashes, the lid wiper, and the blink to distribute the applied solution into the precorneal film. The chosen active ingredient is present in aqueous solution or as a suspension in the substrate of the applicator. Intuitively, one can see wiping is a more physiological method of delivery by contrast to eye drops, which are delivered as a sudden bolus onto the bulbar surface or the conjunctival sac.

By wiping the applicator from the lateral to medial direction, one is taking advantage of the natural direction of tear secretion, distribution, and flow. The eyelid skin and eyelashes serve as a platform and reservoir for the delivered fluid to mimick the properties of secreted tears. By wiping from the lateral to medial direction, one is also taking advantage of the fact that the eyelashes on the lateral half of the eye are slightly longer on average than the medial half, and is therefore a more efficient reservoir.

The solution may also be applied by touching the wet applicator to the lateral canthus with the eyes closed, followed by opening the eyes and blinking. But in this method there is a greater chance of formation of a bolus, and this enlarged drop will then affect the corneal surface and produce irritation.

In summary, delivery of the active ingredient by the swab method is analogous to adding the active ingredient to the windshield wiper fluid and utilizing the blink as the blade to wipe the active ingredient across the eye surface. By contrast, an eye drop delivery method is like putting an active ingredient into a bucket of water and splashing the bucket's contents over the eye surface.

### Selection of Active Ingredient for Delivery Unit

In principle, any ocular drug used as eye drops can be adapted to swabs because of convenience of use. Examples of such ocular drugs include: antihistamines, muscarinic receptor agonists and antagonists, adrenergic receptor agonists and antagonists, anti-inflammatory steroids, antibiotics, non-steroidal anti-inflammatory drugs, analgesics, demulcents [“lubricants”], local anesthetics, medications for glaucoma, and immunosuppressants, etc. A second reason for selecting an ingredient is to relieve ocular discomfort by a soothing and cooling action.

The active pharmaceutical ingredient (API) should be evenly dispersible in a liquid composition so that during manufacture the wipe can be moistened with a constant and uniform solution when produced under clean or sterile conditions. For purposes of formulation, the API should preferably be miscible or soluble in aqueous solutions at neutral pH and/or isotonicity and not adherent as a particle to the absorbent substrate. The aqueous solubility of an API will facilitate meeting requirements of sterility, a unit dose dispenser, uniform dose delivery, and formulations free of preservatives.

Examples of ocular drugs that may be incoproated into swabs include: cyclosporine, antihistamines such as olopatadine, α-adrenergic agonist vasoconstrictors such as phenylephrine, napthazoline, or tetrahydrozoline, anti-inflammatory drugs such as diclofenac, anti-viral agents, antibiotics, and demulcents such as polymer “lubricants” such as carboxymethylcellulose, hypromellose, polyethylene glycol 400, hyaluronan, and propanediol(s). The lubricants increase the elastoviscous properties of the ocular fluids [usually this can be achieved with ophthalmic solutions in the range of 25 to 50 centipoises] and are especially useful for the dry eyes syndrome.

A surprising discovery made here is that an eye wipe, for example a 2″×2″ sterile pad, which carries a cooling agent, can be used as a substrate to deliver the cooling agent to the eyes for long-lasting relief of eye irritation, without side-effects. A second surprise was that certain cooling agents evoked a sense of wetness, accompanied by an improvement in visual acuity. Thus, an eye swab or wipe embodiment carrying a preferred cooling or soothing agent provides a substantial therapeutic improvement over eyedrops. Particularly preferred cooling or soothing agents include those designated CPS-148 and 2-5, further described hereinafter.

Neural Mechanisms of Drug Action:

The surfaces of the eyelids, cornea, and conjunctiva contain sensory nerve endings that respond to thermal stimuli that range from cooling to warmth and to pain. Rostral projections of these nerve endings are to the brainstem dorsolateral nucleus via various branches of the trigeminal nerve. By delivering a cooling agent to the upper and lower eyelids, and to the conjunctiva, it is assumed that activation of nerve endings will then create signals that rostrally in the brainstem interdict signals of irritation and discomfort from other sensory nerves. This “gating” or sensory over-ride of nociceptive inputs provides the basis of antinociceptive or analgesic relief.

Effects Evoked from Eye Surface with Cooling and Soothing Agents:

I have found that an unusual effect obtained with application of CPS-148 to the eye surface was an increased sensation of “wetness”. This effect was seen without increased tear production or irritation and may have special benefit for relieving a sense of dryness on the eye surface.

Design of Eye Wipes:

Pre-moistened towelettes are used in personal care products, for example, to wipe one's hands, to wipe a baby's skin after a diaper change, or to remove make-up on the face and around the eyes (e.g Pond's 6″×8″ Clean Sweep Cleansing and Make-up Remover Towelettes). The design of towelettes is well known to the art and generally each is packaged as a single-use sealed unit. Suitable wrapper materials are those which are relatively vapor impermeable, to prevent drying out of the towelette, and able to form a “peelable” seal. Examples of suitable towelette materials for practicing this invention by using as eye wipes include a polypropylene nonwoven, a rayon-polyester formed fabric, polyethylene terephthalate (PET), or polyester polypropylene blends. An example of a towelette packaging is Walgreens Lens Cleaning Wipe which can be purchased at 120 units per box. A suitable size for an eye wipe is exemplified by Walgreens Sterile Pad, 2″×2″, which can be used to deliver the active ingredient to the eye surface. Using these pads, and based on two wipes per eye, the liquid solution delivered per eye is estimated to be ˜16 μl.

The ocular surface is highly sensitive to soothing and cooling agents. Thus, CPS-148 is clinically effective at ˜8 μg/eye delivered via a 0.5 mg/ml solution.

Therapeutic Indications:

Disorders of the ocular structures in which discomfort is a major symptom can be contemplated for treatment by the compositions and methods of the present discovery, these include, but are not limited to:
blepharitis or inflammation of the eyelids,
dry eye syndrome (keratoconjunctivitis sicca), the inadequate wetting of the ocular surface caused, for example, by inadequate tear secretion or rapid evaporation of tears because of poor tear quality,
conjunctivitis, an inflammation of the conjunctiva that is most commonly caused by allergens, smoke, and pollutants, but may also be caused by bacterial and viral infection, and physical agents such as trauma, wind and sunlight,
keratitis, an inflammation of the cornea, that may be caused by physical trauma, such as cataract surgery or refractive eye surgery, and also by bacterial or viral infection. A corneal abrasion is an injury to the epithelium that is superficial enough not to involve the basement membrane. It occurs mainly after mechanical trauma. A corneal ulcer is a defect that involves the stroma, past Bowman's membrane; the lesion can easily become infected and lead to loss of vision.
iritis (anterior uveitis), an inflammation of the iris, a condition that is rare but associated with considerable pain and inflammation
general eye discomfort, for example, caused by extended wear of contact lenses, by eye strain, by air pollutants, or by excessive exposure to the sun.

The eye swab, spray, or wipe with an agent effective to reduce sensory discomfort of the eye as the single active ingredient may be used as a stand alone analgesic. Alternatively, the liquid composition in the wipe may be combined with other ocular drugs, for example, to reduce irritancy and to imrpove therapy. Examples of such adjunctive ocular drugs are cyclosporine, antihistamines such as olopatadine, α-adrnergic agonist vasoconstrictors such as phenylephrine, napthazoline, or tetrahydrozoline, anti-inflammatory drugs such as diclofenac, anti-viral agents, antibiotics, and polymer “lubricants” such as hypromellose, polyethylene glycol 400, hyaluronan, and propanediol(s). The lubricants increase the elastoviscous properties of the ocular fluids and are especially useful for the dry eyes syndrome.

Novelty: Why are Eye Wipes Better than Eyedrops for the Delivery of Active Agents?

The new observation made here is that, for equivalent concentrations of soothing and cooling agents, eye wipes are effective but eyedrops are not effective because of adverse sensations such as stinging, irritation and pain. I deduce the reasons for this phenomenon as follows:
a) Eye drops are delivered to the naked eyeball and, because of the positioning of the eyedropper, provide a pulse entry of drug onto the corneal surface which is densely innervated by sensory fibers that convey the sensations of pain and irritation. There is still uncertainty if psychophysical experience of coolness or cold can be evoked from the corneal surface; that is, the cornea may only respond to pain and irritation, but is not relieved by coolness. Thus, any cooling agent delivered as an eyedrop is likely to evoke unpleasant sensations upon contact with the nerve endings on the corneal surface before it can elicit cooling on the ocular surface.
b) When an eye wipe is used, the eyelids are kept closed and the drug delivery is to the skin covering the eyeball, the edges of the eyelids, the conjunctiva, and, to a smaller extent, the corneal surface. Sensory nerve endings associated with coolness are known to the present in the skin, the eyelids, and the conjunctiva and it is the activation of these nerve endings that provide the sensations of refreshing coolness and cold.
c) Based on the above considerations, an eye wipe, by comparison to an eye drop, avoids a pulsatile dose to the cornea, is more efficient in delivery of the cooling agent to its relevant target receptors, and the duration of action is longer because of increased contact with the skin above the eyeballs and with the edges of the eyelids. Hence, an eye wipe with a cooling agent will enable the desired response of a soothing and refreshing feeling in the eye without irritation and sting.
d) Quantitative measures illustrate the difference between eyedrops and wipes. If an average eyedrop is 35 μl and two drops are applied per eye, the total is 70 μl. Most of this volume is not delivered because the precorneal space in normal subjects is about ˜7 μl. By contrast each wipe, using the methods described in Example A, delivers ˜16 μl per eye, and the delivery is more localized to the eyelids and conjunctiva so the desired pharmacological effect is obtained.
e) It should be noted that the use of eye wipes to deliver pharmacologically active substances to the ocular surface is a relatively novel concept. A recent review of drug delivery to the eye by Novack [Ophthalmic drug delivery: development and regulatory considerations. Clin. Pharmacol. Therap. 85, 539-543, May, 2009] does not mention this mode of drug transfer.

### Experiment 1.

Ethanol, in sufficient concentration, is an irritant on the human ocular surface. Subjects received different ethanol-water concentrations either in eye drops or via the delivery unit. Subjects were asked to report irritation, stinging, burning sensations, or pain in the 2 to 5 min after application. The intensity of the subjective eye sensation was rated as 0, 1, 2 or 3 with: 0 as no effect; 1 as slight; 2 as a clear-cut effect; and 3 as severe. The results are shown in the Table, with several trials in the same individual. The solutions were coded so the subject did not know the nature of the applied substance.

The results are shown in Table 1. The 5% ethanol solution was clearly aversive as an eye drop, producing stinging and pain upon delivery. When 20% was applied to the eye with a wipe, there were mild burning sensations in two subjects, when the solution entered the eye: but it was considered tolerable and acceptable. None of the sensations were present 5 min after exposure. This experiment shows that the swab method of ethanol delivery is less likely than eye drops to cause eye irritation. For ethanol/water, there is ˜4-fold change in decreased sensitivity to irritation when the swab method is used.

TABLE 1
Comparison of the irritant effects of an ethanol/water solution
administered as eye drops or wiped across the orbital skin
(N = 8 experiments). Irritancy was graded on a scale of 0,
1, 2, or 3: with 0 being no effect and 3 strongly irritating.

| Ethanol Concentration %, wt/vol in | | |
|---|---|---|
| water | Eye drops | Delivery Unit |
| 2.5 | 0 | 0 |
| 5 | +2 | 0 |
| 10 | +3 | 0 |
| 20 | — not tested | +1 |

### Example 2

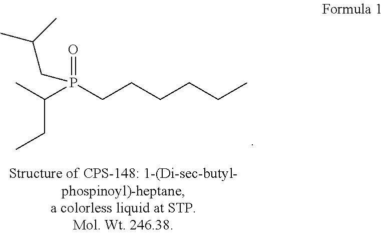 [SMILES: CCCCCCP(=O)(CC(C)C)C(C)CC]

1-(Di-sec-butyl-phosphinoyl)-heptane, coded as CPS-148 (also known as WS-148), is representative of a class of cooling agents called trialkylphosphine oxides [Rowsell and Spring U.S. Pat. No. 4,070,496, Jan. 24, 1978] and was synthesized and tested. Briefly, in the method of synthesis, di-sec-butylphosphinoyl chloride was prepared (as described by R. H. Williams, L. A. Hamilton J. Am. Chem. Soc. (1952), 74, 5418), by addition of sec-butyl magnesium bromide to diethyl phosphite in dry tetrahydrofuran (THF), followed by chlorination in carbon tetrachoride solution at 0-10° C., to yield the di-sec-butylphosphinoyl chloride, which was purified by distillation. A solution of di-sec-butylphosphinoyl chloride (3.9 gm) in dry tetrahydrofuran (50 ml.) was added drop-wise under dry nitrogen to a refluxing solution of n-heptylmagnesium bromide (prepared from magnesium turnings (1.2 gm), n-heptyl bromide (9.0 gm) and dry THF (100 ml.). The mixture was heated under reflux for 18 hours. After cooling to room temperature, the reaction mixture was poured onto ice and 2N HCl (300 ml.), and extracted with methylene dichloride. The combined extracts were washed with lithium hypochlorite solution to oxidize any phosphorus (III) compounds to phosphorus(V) compounds or phosphorus-containing acids, 2N NaOH solution and finally with water, then dried (MgSO₄). The solvent was removed by distillation and the residual yellow oil (8 gm) was eluted with chloroform down a silica gel column. The product (R_{f}=0.1 to 0.2 on silica t.l.c. (CHCl₃)) was finally distilled to yield 1-(Di-sec-butyl-phosphinoyl)-heptane (coded as CPS-148) as a colorless liquid, bp. 120° C.

In a previous study [Wei, Ophthalmic compositions and method for treating eye discomfort and pain. US 2005/0059639A1, Mar. 17, 2005], I had reported the testing of CPS-148 in an eye drop formulation. These procedures were used: A 0.05% (0.5 mg/ml) di-sec-butyl-n-heptyl-phosphine oxide or di-sec-butyl-n-hexyl-phosphine oxide eye drop solution was prepared by adding the compound to an isotonic solution of sodium chloride, 0.65% in deionized water, monobasic potassium phosphate/sodium hydroxide buffer, preserved with disodium EDTA and benzalkonium chloride. The liquid was individually aliquoted into a ¼ oz. bottle with a Yorker spout (E.D. Luce Packaging) suitable for droplet delivery. This solution was applied to the opened eyes of three volunteers, with two to three drops of the solution applied to each eye. The subjects complained of stinging and burning sensations on the eye surface, lasting for about 2 min and requiring the subjects to shut their eyes. Afterwards, the stinging sensations disappeared and were replaced by cooling sensations on the eyelids and eye surfaces lasting for about 1.5 to 2 hours.

The above experiment was repeated at the various concentration of CPS-148, only this time the solution was wiped onto the eyes, using procedures described in Example A. Surprisingly, and to my amazement, there was no irritation and refreshing cooling sensations were obtained from this method of application, although if the residue of the agent was washed onto the eye surface, for example, when taking a shower or wiping the face with a wet towel, irritation was observed. The dose-response data for CPS-148 on cooling sensations in the eye after wipes are shown in FIG. 3.

FIG. 3: “Cooling response of the ocular surface to CPS-148 applied with eye wipes. The dose in μg is per eye, based on solutions of 0.25, 0.5 and 1 mg of CPS-148/ml of saline applied in a volume of 16 μl per eye. At the 8 μg and 16 μg dose cooling was accompanied by a sensation of “wetness”. Even at the high dose there was no sensation of sting, irritation or pain.”

Thus, the use of an indirect method of drug delivery such an eye wipe is a key factor in obtaining the desired pharmacological result: namely, soothing and coolness, without sting and discomfort.

### Example 3

A 75-year old male had a diagnosis of “dry eyes syndrome” of 3 years duration. His principal complaints were of dryness, itch, and discomfort at the corner of his eyes and blurred vision. These symptoms were present on a daily basis at various levels of intensity. He used Systane™ eyedrops but said they were minimally effective. It was not clear if he adhered to a strict regimen of self-administration, but he complained of the eye drops running down his cheeks and the expense of the OTC medication. When the symptoms were severe, the subject found some relief by wetting his eyes with cold tap water, but the relief was only temporary. He volunteered to test an eye wipe containing 0.8 ml of a 0.5 mg/ml solution of CPS-148. Within two minutes the subject reported cooling and soothing sensations on his eyes that were maintained for 1.5 hours. The subject remarked on the sensation of “wetness”, and a feeling of comfort. The subject continues to use the eye wipes for symptomatic relief on an “as-needed basis.”

### Example 4

Sensory discomfort of the eye is a conspicuous symptom in patients diagnosed with “dry eyes disease.” The study described below was conducted by Prof. K. C. Yoon of the Department of Ophthalmology, Chonnam National University Medical School, Gwangju, South Korea. Prof. Yoon is an acknowledged expert on the investigation of dry eyes disease and has published extensively on this subject [e.g. Yoon, K.-C. et al. Application of umbilical cord serum eyedrops for the treatment of dry eye syndrome. Cornea 25, 268-72 (2006). Yoon, K.-C. et al. Comparison of autologous serum and umbilical cord serum eye drops for dry eye syndrome. Am. J. Ophthalmol. 144, 86-92 (2007).] The methods used in these two publications were also used in the experiments described below. This report was presented as an Oral Communication entitled “Effect of a periocular spray of a TRPM8 agonist on the ocular surface” at the 110th Meeting of the Korean Ophthalmological Society, Bexco, Busan, Korea on Apr. 13, 2014.

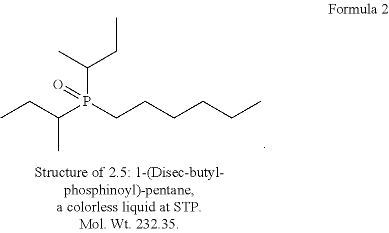 [SMILES: CCCCCCP(=O)(C(C)CC)C(C)CC]

Chemical Synthesis:

The test compound, designated as 2-5 in the figures [1-di(sec-butyl) phosphinoyl-pentane also known as Dapa-8, CAS Registry No. 52911-13-4] was prepared by custom synthesis at Phoenix Pharmaceuticals, Burlingame, Calif., USA. The method of synthesis was: 100 mL (23.7 g, ˜200 mmol) of sec-butylmagnesium bromide, obtained from Acros, as a 25% solution in tetrahydrofuran (THF)), was placed under nitrogen in a 500 mL flask (with a stir bar). Diethylphosphite solution in THF (from Aldrich, D99234; 8.25 g, 60.6 mmol in 50 mL) was added drop-wise. After approximately 30 minutes, the reaction mixture warmed up to boiling. The reaction mixture was stirred for an extra 30 minutes, followed by a drop-wise addition of n-pentane iodide solution in THF (from TCI; 60 mmol in 20 mL). The reactive mixture was then stirred overnight at room temperature. The reaction mixture was diluted with water, transferred to a separatory funnel, acidified with acetic acid (˜10 mL), and extracted twice with ether. The ether layer was washed with water and evaporated (RotaVap Buchi, bath temperature 40° C.). The light brown oil was distilled under high vacuum. The final product, verified by mass as determined by mass spectrometry, was a clear, colorless liquid.

2-5 was prepared as a 2 mg/mL solution in distilled water and administered as a spray [sprayed volume of ˜0.1 mL] over the closed right eye of test subjects. There were 20 subjects in the normal group [9 males/11 females] and 17 subjects in the group with defined “dry eyes syndrome” [7 males/10 females]. Exclusion criteria for patient selection was age of <18 years, systemic medication that may affect tear secretion, previous punctal plug insertion, history of contact lenses use, previous ocular surgery or trauma, pregnancy or lactation, and ocular or systemic disease. The average age of the subjects was 29 years, and both groups had normal values for visual acuity, intraocular pressure, and corneal sensitivity [Cochet-Bonnet esthesiometry, measured with a microfilament]. In the “dry eyes syndrome” group, however, the Breakup Time of tears [BUT, sec], Schirmer I test [mm] were significantly impaired: BUT 11.1±2.1 vs 5.5±1.5 [sec] and Schirmer I of 14.1±3.5 vs 5.1±1.1 [mm]. The score for keratoepitheliopathy [National Eye Institute system] was 0.1±0.3 and 2.5±1.7, in the normal vs the “dry eyes syndrome” group.

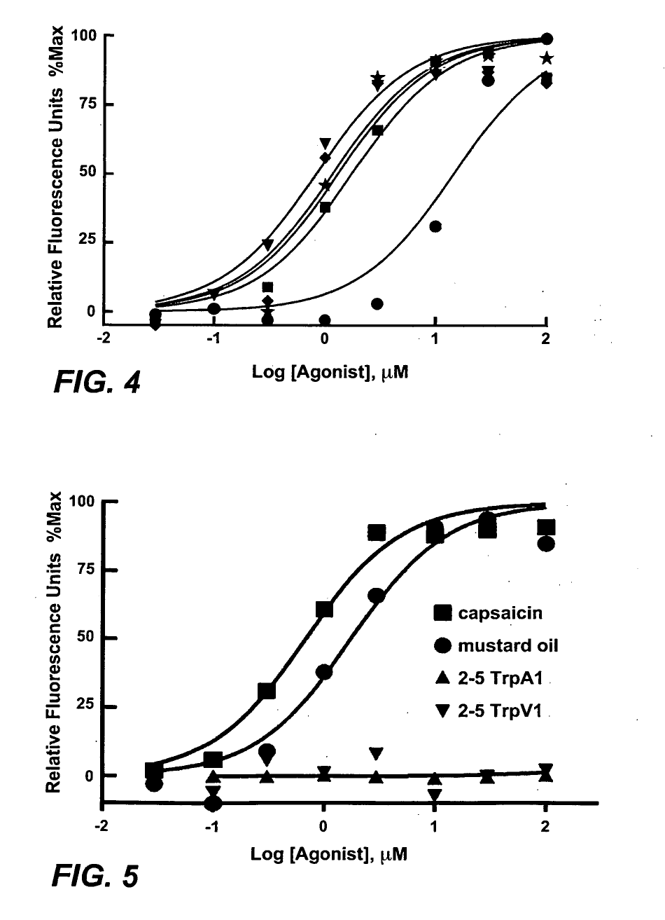

FIG. 4 is a graph of fluorescence (Relative Fluourescence Units; % Maximum) of test compounds, as a function of the logarithm of the concentration of the test compound (μM). The compounds tested were 2-4 (circle), 2-5 (square), 2-6 (inverted triangle), 2-7 (diamond), and 2-8 (star), representing the 1 [di-sec-butyl-phosphinoyl]-butane, pentane, hexane, heptane, and octane analogs, respectively. The receptor assays were conducted by ChanTest Corporation, Cleveland, Ohio 44128. 2-4 is significantly less potent than 2-5, 2-6, 2-7, and 2-8. The 95% confidence intervals of 2-5 to 2-8 overlap. There are, however, distinct, selective pharmacological differences among these compounds when administered in vivo, 2-5 being the compound with minimal irritation when applied to the skin of the eyelids. The potencies of these analogs, relative to 1-menthol in the same assay, are for 2-4 [0.3×], 2-5 [2.2×], 2-6 [4-9×], 2-7 [3.8×], and 2-8 [3.0×].

FIG. 5. illustrates the activity of 2-5 on cells transfected with the TRPA1 and TRPV1. TRPA1 and TRPV1 receptors mediate nociception: that is, stimulation of these receptors results in irritation and pain. Capsaicin and mustard oil are the positive control substances for TRPV1 and TRPA1, respectively. It can be seen that 2-5 does not stimulate TRPA1 and TRPV1, and is thus a selective TRPM8 receptor agonist.

Subjective symptoms were graded on a numerical score. A visual analog scale score [0 to 10 units] was used for grading the degree of soothing and cooling sensation on the ocular surface. For symptoms of the dry eyes syndrome, a scale of zero to four was used, with zero representing no symptoms and four representing very severe symptoms that caused discomfort and interfered with normal activities. These methods have been described in detail elsewhere and are incorporated by reference [Yoon, K.-C. et al. Application of umbilical cord serum eyedrops for the treatment of dry eye syndrome. Cornea 25, 268-72 (2006). Yoon, K.-C. et al. Comparison of autologous serum and umbilical cord serum eye drops for dry eye syndrome. Am. J. Ophthalmol. 144, 86-92 (2007)].

After delivery of 2-5 to the ocular surface by spraying the closed eye, all subjects felt soothing and cooling sensations on the ocular surface after blinking. The onset was less than 3 min and reached maximal effect in the first 10 min. The mean duration of cooling was 42±8 min and all cooling sensations disappeared by 1 hr. For patients previously diagnosed with “dry eyes” disease there was a reduction of symptom score from 1.8 to 0.5, a highly significant event [P<0.001]. The Tear Film BUT and tear secretion rates were not significantly affected in the normal group, and slightly elevated in the first 30 min in the dry eyes group. However, the magnitude of the effect was not considered clinically significant and may have been due to increased sensitivity of the dry eyes patients to foreign solutions applied to the ocular surface. The keratoepitheliopathy score was not affected by treatment.

The mechanical sensitivity of the cornea, measured with the Cochet-Bonnet esthesiometer, was not affected in all patients, in the 1.5 hr test period with 5 points of measurement, spaced 30 min apart. This is an important observation from the viewpoint of safety in use, because numbness of the corneal surface will increase risks of eye injury from foreign materials.

These experiments provide the first direct human evidence that the sensory discomfort experienced by patients diagnosed with “dry eyes syndrome” can be relieved by topical applications of a trialkyl phosphine oxide cooling agent. The indirect method of drug delivery to the ocular surface is a key factor for a successful demonstration of this drug action.

1. An apparatus for treating eye discomfort, comprising:
a pharmaceutically active agent, the agent providing a soothing or cooling effect to reduce sensory discomfort when topically applied to a body surface in contact with air; and,
a substrate adapted to deliver the agent to skin covering the eyeball, the edges of the eyelids, and the conjunctiva when contacted therewith.

2. The apparatus as in claim 1 wherein the agent is selected from the group consisting of trialkylphosphine oxides.

3. The apparatus as in claim 1 wherein the agent is 1-di(sec-butyl) phosphinoyl-heptane.

4. The apparatus as in claim 1 wherein the agent is 1-di(sec-butyl) phosphinoyl-pentane.

5. A method of treating eye discomfort comprising;
providing an eye wipe carrying an agent that is a liquid at standard room temperature and pressure; and
contacting the skin covering the eyeball, the edges of the eyelids, and the conjunctiva with the eye wipe.

6. The method as in claim 6 wherein the agent is selected from the group consisting essentially of trialkylphosphine oxides.

7. The method as in claim 6 wherein the agent is 1-[Di-sec-butyl phosphinoyl]-heptane.

8. The method as in claim 6 wherein the agent is 1-[Di-sec-butyl phosphinoyl]-pentane.

9. An ocular drug delivery unit, comprising:
an applicator, the applicator having a handle and a liquid absorbent portion, the absorbent portion being spaced from the handle, the absorbent portion having an absorbent mass of from about 40 to 100 mg; and,
a reservoir of an ocular drug in liquid form, the drug being of sufficient quantity as to saturate the absorbent mass of the applicator when contacted therewith.

10. The delivery unit as in claim 9 wherein the absorbent portion, when saturated with ocular drug from the reservoir, is of sufficient construction so as to off-load 8 to 20 μL of the ocular drug per eye when topically applied to the lower half of an upper eyelid over the lashes.

11. The delivery unit as in claim 9 wherein the ocular drug is selected from a trialkylphosphine oxide consisting of 1-[Di-sec-butyl phosphinoyl]-heptane, 1-[Di-sec-butyl phosphinoyl]-pentane, and mixtures thereof.

12. The delivery unit as in claim 11 wherein the ocular drug is 1-[Di-sec-butyl phosphinoyl]-heptane.

13. The delivery unit as in claim 11 wherein the ocular drug is 1-[Di-sec-butyl phosphinoyl]-pentane.

14. A kit for use with eye disorders, comprising:
an applicator adapted to deliver a predetermined dose of a drug or liquid to one or both upper eyelids, wherein the applicator comprises a handle and an absorbent material of predetermined mass carried by the handle;
a quantity of drug in solution sufficient to saturate the absorbent material; and,
instructions for preparation and use of the applicator and drug.

15. The kit as in claim 14 wherein the absorbent material is cotton or rayon.

16. The kit as in claim 14 wherein the mass of the absorbent material is about 40 to 100 mg.

17. The kit as in claim 14 wherein the drug is selected from the group of trialkyl phosphine oxides.

18. The kit as in claim 14 wherein the ocular drug is 1-[Di-sec-butyl phosphinoyl]-heptane.

19. The kit as in claim 14 wherein the ocular drug is 1-[Di-sec-butyl phosphinoyl]-pentane.
